(19) United States
(12) Patent Application Publication
(10) Pub. No.: US 2026/0240728 A1
(43) Pub. Date: Aug. 20, 2026

(54) Potion Brush

(71) Applicants: Paula Berends, Elgin, SC (US); Shamene Jones, Elgin, SC (US)

(72) Inventors: Paula Berends, Elgin, SC (US); Shamene Jones, Elgin, SC (US)

(21) Appl. No.: 19/360,594

(22) Filed: Oct. 16, 2025

Related U.S. Application Data
(60) Provisional application No. 63/760,191, filed on Feb. 19, 2025.

(51) Int. Cl. A61K 8/02; A46B 11/00; A61Q 5/12

(52) U.S. Cl. CPC A61K 8/0216; A46B 11/0068; A46B 11/0072; A61Q 5/12; A46B 2200/104; A61K 2800/87

(57) ABSTRACT

A hairbrush containing a solid, water soluble hair conditioner. As the hairbrush is moved through the hair, the conditioner is dispersed through the perforations (holes) alongside the hairbrush's bristles.

### CROSS REFERENCE TO RELATED APPLICATIONS

None

### STATEMENT REGARDING FEDERALLY SPONSORED RESEARCH OR DEVELOPMENT

This invention was not made by an agency of the United States Government nor under a contract with an agency of the United States Government.

### THE NAME OF THE PARTIES TO JOINT RESEARCH AGREEMENT

Not Applicable.

### INCORPORATION BY REFERENCE OF MATERIAL SUBMITTED ON A COMPACT DISC OR AS A TEXT FILE VIA THE OFFICE ELECTRONIC FILING SYSTEM (EFS-WEB)

Not Applicable.

### STATEMENT REGARDING PRIOR DISCLOSURES BY THE INVENTOR OR A JOINT INVENTOR

Provisional Patent Application: 63/760,191
Filing Date: Feb. 19, 2025

### BACKGROUND OF THE INVENTION

### Field of the Invention

Natural oils have been used for centuries to condition human hair. Modern hair conditioner was created at the beginning of the 20th century with the introduction of a product at the 1900 World's Fair.

Conditioners are available today in a wide variety of forms including solids, viscous liquids, gels, creams, lotions, and sprays. Hair conditioner is typically applied to hair following the hair being washed and after a short period it may either be rinsed out or left in.

The present invention involves the use of a water soluble solid hair conditioner that is inside a hairbrush where the brush is equipped with holes to allow water to come into contact with the bar and for the conditioner to exit the interior of the brush and be dispersed into the hair by the movement of the brush's bristles through the hair.

### Description of Related Art

A number of US patents and published patent applications disclose formulations for solid hair conditioners. Among them are U.S. Pat. No. 10,799,438 (Constantine, et al), U.S. Pat. No. 6,831,046 (Carew, et al), US Patent Application Publication 202404157744 (Story), and US Patent Application Publication 20220273528 (Scheele, et al). However, none of these inventions disclose a solid hair conditioner contained in a brush for ease if distribution of the conditioner throughout the hair.

### BRIEF SUMMARY OF THE INVENTION

The present invention is a hairbrush containing a solid, water soluble hair conditioner. As the brush is moved through the hair, the conditioner is dispersed through the holes near the brush's bristles.

### BRIEF DESCRIPTION OF THE DRAWING

FIG. 1 is a view of the front (bristle side) of the invention.

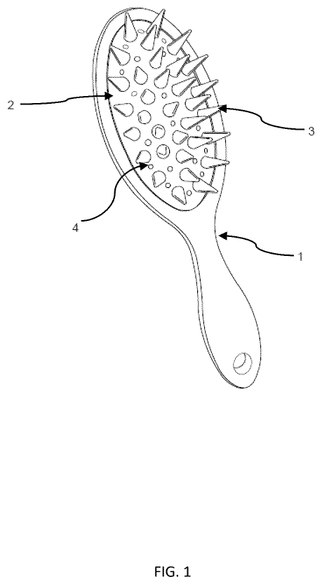

FIG. 2 is a view of the back of the invention.

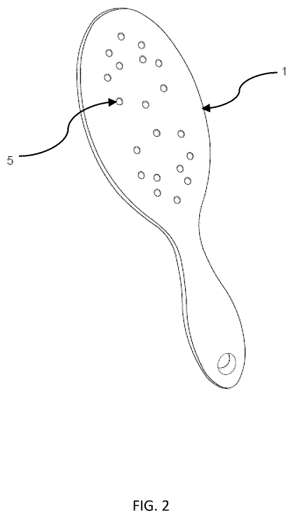

### DETAILED DESCRIPTION OF THE INVENTION

As used in this application, a hairbrush is defined as a brush for use on the human hair with bristles on one side of the brush. A solid is defined as a substance that holds its shape without packaging support and product release is triggered by the addition of water.

The present invention is a hairbrush containing a solid, water soluble hair conditioner. As the hairbrush is moved through the hair, the conditioner is dispersed through the perforations (holes) alongside the hairbrush's bristles. The body (1) of the hairbrush including the handle is made of acrylonitrile butadiene styrene (ABS) and the bristle side (2) of the hairbrush including the bristles (3) is made of Thermoplastic rubber (TPR). TPR is a blend of polymers and elastomers.

A solid hair conditioner is located below the soft section (2) of the hairbrush. Water is allowed to enter and come into contact with the solid conditioner via perforations (5) in the back of the hairbrush. The solubilized conditioner and water can then exit the hairbrush via the holes (4) in the bristle side (2) of the hairbrush where it is dispersed through the hair by the bristles (3) in the act of brushing the hair.

The brush can come in a variety of colors, with the color indicative of the formulation of the solid conditioner. Two different formulations for the solid conditioner contained within the dispersing brush are provided in Tables 1 and 2.

TABLE 1
Proportions of Ingredients in One of the
Preferred Embodiments of the Invention

| Ingredient | % By Weight | % Weight Range |
|---|---|---|
| Varisoft ® EQ 65 | 15 | 10-20 |
| MCT/Jojoba Esteris-70 | 32 | 27-37 |
| Glyceryl Stearate SE | 3.2 | 1-5 |
| Jojoba Oil | 15 | 10-20 |
| Propanediol | 10 | 5-15 |
| Glycerine | 5 | 1-10 |
| Panethenol | 1.5 | 0.5-2.5 |
| Betaine | 2.5 | 1-4 |
| Pentylene Glycol | 3 | 1-5 |
| Lecithin | 2 | 1-3 |
| Sorbitol | 2.5 | 1-4 |
| Siligel | 2.5 | 1-4 |
| Tapioca Starch | 1.5 | 0.5-2.5 |
| Citric Acid | 0.3 | 0.1-1 |
| Euxyl K903 | 1.0 | 0.1-2 |
| Tocopherol | 0.5 | 0.1-1 |
| Fragrance | 0.5 | 0.1-1 |
| Amino Acid | 2.0 | 1-3 |
| Total: | 100 | |

TABLE 2
Proportions of Ingredients in One of the
Preferred Embodiments of the Invention

| Ingredient | % By Weight | % Weight Range |
|---|---|---|
| Varisoft ® EQ 65 | 14.5 | 10-20 |
| MCT/Jojoba Esteris-70 | 30.7 | 27-37 |
| Coco Caprylate | 6 | 1-11 |
| Cetyl Alcohol | 9.5 | 5-15 |
| Propanediol | 6.5 | 1-12 |
| Pentylene Glycol | 1 | 0.1-2 |
| Siligel | 2 | 1-3 |
| Glycerine | 5 | 1-10 |
| Macerated Jojoba with herbs | 8 | 3-13 |
| Panethenol | 2 | 1-3 |
| Betaine | 2 | 1-3 |
| Amino Acid | 3 | 1-5 |
| Sorbitol | 6 | 1-10 |
| Citric Acid | 0.3 | 0.1-1 |
| Euxyl K903 | 1.0 | 0.1-2 |
| Tapioca Starch | 1.5 | 0.1-3 |
| Essential Oil Blend | 1.0 | 0.1-2 |
| Total: | 100 | |

Varisoft® EQ 65 is a natural, biodegradable cationic emulsifier and surfactant. It is comprised of Distearoylethyl Dimonium Chloride (and) Cetearyl Alcohol

MCT/Jojoba Esteris 70 is an blend with 85% medium chain triglycerides (MCT) and 15% jojoba esteris 70. Medium chain triglycerides are fat molecules usually made from coconut and palm kernel oils. MCT is used for its penetrating ability to the hair shaft, where it helps strengthen hair, protect hair from damage, and prevent protein loss. Jojoba Esteris-70 is an oil-free wax ester derived from jojoba oil or hydrogenated jojoba oil. It provides hydration and non-greasy emolliency.

Glyceryl Stearate SE is a combination of glycerin and stearic acid. The SE stands for self-emulsifying, indicating that it contains a small amount of potassium or sodium stearate, which enhances its emulsifying capabilities. Glyceryl Stearate SE also provides moisturizing effects, helping to hydrate the hair and scalp. It also contributes to the stability of formulations, enhancing shelf life.

Jojoba oil is a natural oil known for its moisturizing properties. It is extracted from the seeds of the jojoba plant.

Propanediol (also called 1,3-propanediol) is a natural solvent and emollient that's derived from corn sugar. Propanediol acts as a humectant, helping to keep the skin hydrated by attracting moisture. It also enhances the absorption of other active ingredients in formulations, thus increasing their effectiveness.

Glycerine, also known as glycerol, is commonly found in skin and hair care products due to its moisturizing properties.

Panthenol is a chemical substance made from pantothenic acid, also known as vitamin B-5. It is used in hair care products because of its ability to improve hair's shine, softness, and strength.

Betaine (trimethylglycine), is a naturally occurring amino acid that enhances moisture retention and combats hair damage. In hair conditioners, betaine acts as a humectant, helping to maintain long-lasting hydration. It also helps maintain the hair's natural barrier, protecting it from water loss, allergens and bacteria.

Pentylene glycol is used in hair care products as it provides several benefits. Its humectant properties helps hair retain moisture. It aids in dissolving both water soluble and oil soluble ingredients helping with the even distribution of other active ingredients. Pentylene glycol also acts as a stabilizer and preservative.

Lecithins are mixtures of glycerophospholipids, including phosphatidylcholine, phosphatidylethanolamine, phosphatidylinositol, phosphatidylserine, and phosphatidic acid. Lecithin provides several benefits for the hair. It acts as a moisturizer. It improves hair texture by forming a protective barrier around the hair and can help reverse damage to the hair caused by styling, chemical treatments, and environmental factors.

Sorbitol, also known as glucitol, is a sugar alcohol with a chemical formula of C₆H₁₄O₆. It is a common ingredient in hair care products due to its humectant properties, which help draw moisture from the environment and retain it within the hair and scalp.

Siligel is a natural gelling agent that can be used as a gelling agent, stabilizer, co-emulsifier, or suspending agent.

Tapioca starch is a versatile ingredient in hair care, known for its oil-absorbing properties and ability to add volume.

Citric acid is used in hair care products, to balance pH, enhance hair shine, and promote scalp health. Citric acid exfoliating properties helps remove dead skin cells from the scalp, promoting healthier hair growth and reducing dandruff.

Euxyl K 903 is a liquid preservative used in personal care products, particularly effective against bacteria, yeast and molds. Suitable for both leave-on and rinse-off products.

Tocopherol is one of the forms of Vitamin E.

Amino acids are used to strengthen hair and reduce hair frizz.

Coco Caprylate is used both for its moisturizing benefits and its lightweight feel on the skin. It is derived from coconut oil.

Cetyl Alcohol, also known as hexadecane-1-ol, used as an emollient (softens or sooths the skin), emulsifier and thickening agent.

The macerated jojoba with herbs is a mixture of 10 parts jojoba oil to 1 part each of rosemary, thyme, comfrey root, horsetail, and lavender. Each of these herbs are known for their potential to promote hair growth.

The essential oil blend is comprised of equal parts sweet orange oil, rosemary oil, rose geranium oil, patchouli oil, and ginger oil. Sweet orange oil provides a fresh citrus scent and is used for its cleansing and refreshing qualities. Rosemary essential oil is traditionally used in hair care to help maintain a healthy looking scalp and strong, vibrant hair. Rose geranium essential oil contributes a floral scent and is known for helping to keep the scalp comfortable and hydrated. Patchouli essential oil is known for its rich, earthy aroma and is used to help balance scalp oils, supporting both dry and oily hair types. Ginger essential oil provides a warm, spicy fragrance and is included in hair products to invigorate and refresh the scalp, supporting the look and feel of healthy hair.

The present invention described above is visualized as the preferred embodiment of the invention. It is envisioned that this invention may be embodied in many different forms and should not be construed as limited to the embodiments set forth herein. It will be understood by those skilled in the art that changes in details may be made without departing from the spirit and scope of the present application.

Suggested classifications for this invention include:
A46B 11/100: Brushes with reservoirs or other means of applying substances;
A46B 2200/104: Hairbrush;
A61K 8/0216: Cosmetics or similar toiletry preparations, solid or semi-solid forms;
A61Q 5/12: Preparations for care of the hair, Preparations containing hair conditioners.

The terminology used herein is for describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated elements, and/or components, but do not preclude the presence or addition of one or more other elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The invention claimed is:

1. A hairbrush containing a solid hair conditioner where said hairbrush has perforations on both the non-bristle and bristle sides to allow the entry of water and the release of said hair conditioner to allow distribution of the hair conditioner throughout the hair.

2. The solid hair conditioner of claim 1, where said solid hair conditioner consists of 15% Varisoft® EQ65, 32% MCT/Jojoba Esteris-70, 3.2% glyceryl stearate SE, 15% jojoba oil, 10% propanediol, 5% glycerine, 1.5% panethenol, 2.5% betaine, 3% pentylene glycol, 2% lecithin, 2.5% sorbitol, 2.5% Siligel, 1.5% tapioca starch, 0.3% citric acid, 1% Euxyl K903, 0.5% tocopherol, 0.5% fragrance, and 2% amino acids.

3. The solid hair conditioner of claim 1, where said solid hair conditioner consists of 10-20% Varisoft® EQ65, 27-37% MCT/Jojoba Esteris-70, 1-5% glyceryl stearate SE, 10-20% jojoba oil, 5-15% propanediol, 1-10% glycerine, 0.5-2.5% panethenol, 1-4% betaine, 1-5% pentylene glycol, 1-3% lecithin, 1-4% sorbitol, 1-4% Siligel, 0.5-2.5% tapioca starch, 0.1-1% citric acid, 0.1-2% Euxyl K903, 0.1-1% tocopherol, 0.1-1% fragrance, and 1-3% amino acids.

4. The solid hair conditioner of claim 1, where said solid hair conditioner consists of 14.5% Varisoft® EQ65, 30.7% MCT/Jojoba Esteris-70, 6% coco caprylate, 9.5% cetyl alcohol, 6.5% propanediol, 1% pentylene glycol, 2% Siligel, 5% glycerine, 8% macerated jojoba with herbs (67% jojoba oil and 33% herbs where said herbs are equal parts rosemary, thyme, comfrey root, horsetail, and lavender), 2% panethenol, 2% betaine, 3% amino acid, 6% sorbitol, 0.3% citric acid, 1% Euxyl K903, 1.5% tapioca starch and 1% essential oil blend (equal parts sweet orange oil, rosemary oil, rose geranium oil, patchouli oil, and ginger oil).

5. The solid hair conditioner of claim 1, where said solid hair conditioner consists of 10-20% Varisoft® EQ65, 27-37% MCT/Jojoba Esteris-70, 1-11% coco caprylate, 5-15% cetyl alcohol, 1-12% propanediol, 0.1-2% pentylene glycol, 1-3% Siligel, 1-10% glycerine, 3-13% macerated jojoba with herbs (67% jojoba oil and 33% herbs where said herbs are equal parts rosemary, thyme, comfrey root, horsetail, and lavender), 1-3% panethenol, 1-3% betaine, 1-5% amino acid, 1-10% sorbitol, 0.1-1% citric acid, 0.1-2% Euxyl K903, 0.1-3% tapioca starch and 0.1-2% essential oil blend (equal parts sweet orange oil, rosemary oil, rose geranium oil, patchouli oil, and ginger oil).

6. The hairbrush of claim 1, where said hairbrush is constructed of acrylonitrile butadiene styrene and thermoplastic rubber.

7. A hairbrush containing a solid hair conditioner where said hairbrush has perforations on both the non-bristle and bristle sides to allow the entry of water and the release of said hair conditioner to allow distribution of the hair conditioner throughout the hair and where said hairbrush is constructed of acrylonitrile butadiene styrene and thermoplastic rubber.

8. The solid hair conditioner of claim 7, where said solid hair conditioner consists of 15% Varisoft® EQ65, 32% MCT/Jojoba Esteris-70, 3.2% glyceryl stearate SE, 15% jojoba oil, 10% propanediol, 5% glycerine, 1.5% panethenol, 2.5% betaine, 3% pentylene glycol, 2% lecithin, 2.5% sorbitol, 2.5% Siligel, 1.5% tapioca starch, 0.3% citric acid, 1% Euxyl K903, 0.5% tocopherol, 0.5% fragrance, and 2% amino acids.

9. The solid hair conditioner of claim 7, where said solid hair conditioner consists of 10-20% Varisoft® EQ65, 27-37% MCT/Jojoba Esteris-70, 1-5% glyceryl stearate SE, 10-20% jojoba oil, 5-15% propanediol, 1-10% glycerine, 0.5-2.5% panethenol, 1-4% betaine, 1-5% pentylene glycol, 1-3% lecithin, 1-4% sorbitol, 1-4% Siligel, 0.5-2.5% tapioca starch, 0.1-1% citric acid, 0.1-2% Euxyl K903, 0.1-1% tocopherol, 0.1-1% fragrance, and 1-3% amino acids.

10. The solid hair conditioner of claim 7, where said solid hair conditioner consists of 14.5% Varisoft® EQ65, 30.7% MCT/Jojoba Esteris-70, 6% coco caprylate, 9.5% cetyl alcohol, 6.5% propanediol, 1% pentylene glycol, 2% Siligel, 5% glycerine, 8% macerated jojoba with herbs (67% jojoba oil and 33% herbs where said herbs are equal parts rosemary, thyme, comfrey root, horsetail, and lavender), 2% panethenol, 2% betaine, 3% amino acid, 6% sorbitol, 0.3% citric acid, 1% Euxyl K903, 1.5% tapioca starch and 1% essential oil blend (equal parts sweet orange oil, rosemary oil, rose geranium oil, patchouli oil, and ginger oil).

11. The solid hair conditioner of claim 7, where said solid hair conditioner consists of 10-20% Varisoft® EQ65, 27-37% MCT/Jojoba Esteris-70, 1-11% coco caprylate, 5-15% cetyl alcohol, 1-12% propanediol, 0.1-2% pentylene glycol, 1-3% Siligel, 1-10% glycerine, 3-13% macerated jojoba with herbs (67% jojoba oil and 33% herbs where said herbs are equal parts rosemary, thyme, comfrey root, horsetail, and lavender), 1-3% panethenol, 1-3% betaine, 1-5% amino acid, 1-10% sorbitol, 0.1-1% citric acid, 0.1-2% Euxyl K903, 0.1-3% tapioca starch and 0.1-2% essential oil blend (equal parts sweet orange oil, rosemary oil, rose geranium oil, patchouli oil, and ginger oil).
